(19) United States
(12) Patent Application Publication
(10) Pub. No.: US 2024/0158583 A1
(43) Pub. Date: May 16, 2024

(54) BIO-BASED HYDROGELS AND METHOD OF MAKING THE SAME

(71) Applicant: Technology Innovation Institute - Sole Proprietorship LLC, Masdar City (AE)

(72) Inventors: Natasa Tomic, Masdar City (AE); Abdullahi Naseer Mustapha, Masdar City (AE); Maitha Almheiri, Masdar City (AE); Nujood Saeed Ali Mohammed AlShehhi, Masdar City (AE)

(73) Assignee: Technology Innovation Institute - Sole Proprietorship LLC, Masdar City (AE)

(21) Appl. No.: 18/508,697

(22) Filed: Nov. 14, 2023

Related U.S. Application Data
(60) Provisional application No. 63/383,937, filed on Nov. 16, 2022.

(51) Int. Cl. C08J 3/075; A61B 5/00; C08K 3/04; C08K 9/04; C08K 9/08

(52) U.S. Cl. CPC C08J 3/075; A61B 5/6801; C08K 3/042; C08K 9/04; C08K 9/08; C08J 2333/26; C08K 2201/011

(57) ABSTRACT

The present disclosure describes conductive hydrogels which may include a natural polysaccharide for improved performance and biocompatibility. The resulting conductive hydrogel may exhibit high conductivity and stretchability along with excellent self-healing capability. Methods of producing such hydrogels are also disclosed.

### CROSS-REFERENCE TO RELATED APPLICATIONS

This application claims priority to U.S. Provisional Patent Application No. 63/383,937, which was filed on Nov. 16, 2022, the entire contents of which are incorporated by reference herein.

### FIELD

The present disclosure relates generally to hydrogels formed from bio-based materials, and methods of making and using the same. More specifically, the present disclosure relates to bio-based hydrogels which may be conductive and bio-compatible and exhibit self-healing properties.

### BACKGROUND

Hydrogels are three-dimensional (3D) networks of hydrophilic polymer cross-linked with varying quantities of water. Because hydrogels typically exhibit excellent biocompatibility and hydrophilic characteristics, they are useful in a wide range of applications, including as nanofillers, self-healing materials, strain sensors, wound dressings, and wearable devices.

Conductive hydrogels are desirable targets for many of these applications; however, the conductive polymers which are typically incorporated into hydrogels are often hydrophobic and incompatible with hydrophilic polymer matrices, resulting in uneven dispersion and inadequate conductive performance. Other conductive components such as metal nanoparticles, carbon-based nanomaterials, and graphene have also been incorporated into hydrogels, though homogenous dispersion of the conductive component within the hydrogel remains a challenge.

The combination of self-healing capability with conductivity allows an even wider range of applications to be realized, such as artificial skin and similar designs. A conductive hydrogel wound dressing would offer healing benefits along with the ability to monitor health parameters, and the introduction of self-healing capacity would allow such a device to be reused many times. It is thus of great interest to develop conductive hydrogels with excellent biocompatibility, strong electrochemical performance, and robust self-healing properties.

### SUMMARY

In aspects, there is provided a conductive hydrogel, including: about 5 wt. % to about 100 wt. % polysaccharide, wherein the conductive hydrogel exhibits a conductivity of about 1 mS/cm to about 85 mS/cm and a stretchability of about 1000% to about 5500%, and wherein the conductive hydrogel exhibits self-healing.

In aspects, the polysaccharide includes apple fibers, xanthan gum, Arabic gum, sodium carboxymethyl cellulose, konjac gum, guar gum, microfibrillated cellulose, gelatin, alginate, or combinations thereof. In aspects, the conductive hydrogel according to any of the above aspects includes about 10 wt. % to about 30 wt. % polysaccharide. In aspects, the conductive hydrogel according to any of the above aspects includes about 50 wt. % to about 100 wt. % polysaccharide.

In aspects, the conductive hydrogel according to any of the above aspects further includes about 1 wt. % to about 5 wt. % of a filler relative to polysaccharide content. In aspects, the filler according to any of the above aspects includes activated carbon black, reduced graphene oxide, graphene nanoplatelets, or combinations thereof. In aspects, the conductive hydrogel according to any of the above aspects further includes about 2 wt. % to about 15 wt. % of a crosslinker relative to polysaccharide content. In aspects, the crosslinker according to any of the above aspects includes citric acid, borax, calcium chloride, or combinations thereof. In aspects, the conductive hydrogel according to any of the above aspects further includes about 5 wt. % to about 95 wt. % polyvinyl alcohol, polyacrylamide, polyurethane, poly(acrylic acid), poly(methacrylic acid), poly(3,4-ethylenedioxythiophene)-poly(styrene sulfonate), or combinations thereof.

In aspects, the conductive hydrogel according to any of the above aspects exhibits a conductivity of about 50 mS/cm to about 85 mS/cm. In aspects, the conductive hydrogel according to any of the above aspects exhibits a stretchability of about 3000% to about 5500%. In aspects, the conductive hydrogel according to any of the above aspects exhibits a storage modulus of about 15 kPa to about 60 kPa over a plurality of alternating low-high shear strain cycles.

In aspects, there is provided a device, including the conductive hydrogel according to any of the above aspects, wherein the device functions as a strain sensor, a wound dressing, a health monitor, a self-healing material, or combinations thereof. In aspects, the device according to any of the above aspects is connected to a computer.

In aspects, there is provided a method of making a conductive hydrogel, including: providing a polysaccharide, dissolving the polysaccharide in water to form a solution, heating the solution, and adding a crosslinker to the solution to form a hydrogel.

In aspects, the polysaccharide includes apple fibers, xanthan gum, Arabic gum, sodium carboxymethyl cellulose, konjac gum, guar gum, microfibrillated cellulose, gelatin, alginate, or combinations thereof. In aspects, the method according to any of the above aspects further includes dissolving a polymer in the solution, wherein the polymer includes polyvinyl alcohol, polyacrylamide, polyurethane, poly(acrylic acid), poly(methacrylic acid), poly(3,4-ethylenedioxythiophene)-poly(styrene sulfonate), or combinations thereof. In aspects, heating the solution according to any of the above aspects includes heating the solution to a temperature of about 50° C. to about 100° C. In aspects, the method according to any of the above aspects further includes adding a filler to the solution, wherein the filler includes activated carbon black, reduced graphene oxide, graphene nanoplatelets, or combinations thereof. In aspects, the crosslinker according to any of the above aspects includes citric acid, borax, calcium chloride, or combinations thereof. In aspects, the method according to any of the above aspects further includes treating the hydrogel, wherein treating the hydrogel includes freeze drying the hydrogel, contacting the hydrogel with a solution, or combinations thereof. In aspects, the solution according to any of the above aspects includes lithium chloride, potassium chloride, sulfuric acid, potassium hydroxide, sodium sulfate, or combinations thereof.

### DRAWINGS

Aspects, features, benefits, and advantages of the embodiments described herein will be apparent with regard to the following description, appended claims, and accompanying drawings where:

FIG. 1A, FIG. 1B, and FIG. 1C are a set of images of a conductive hydrogel according to an embodiment of the present disclosure, showing that the conductive hydrogel may be molded into a variety of shapes.

FIG. 2 is a bar graph showing the conductivity for various conductive hydrogels, according to embodiments of the present disclosure.

FIG. 3A and FIG. 3B are a set of images of a conductive hydrogel according to an embodiment of the present disclosure, demonstrating that the conductive hydrogel shown in FIG. 3A may be stretched to over 5000% its original length as shown in FIG. 3B.

FIG. 4A, FIG. 4B, FIG. 4C, and FIG. 4D are plots demonstrating the self-healing ability of conductive hydrogels according to embodiments of the present disclosure, wherein shear strain is alternated between 1% and 200% over five cycles.

FIG. 5 is a flow chart of a method of making a conductive hydrogel, according to an embodiment of the present disclosure.

FIG. 6A shows particle size distribution of samples according to embodiments of the present disclosure. FIG. 6B shows XRD patterns of three samples (ACB, GNP, and rGO) according to embodiments of the present disclosure.

FIG. 7A and FIG. 7B are Nyquist diagrams of conductive hydrogels according to embodiments of the present disclosure.

### DETAILED DESCRIPTION

There is a provided a conductive hydrogel which may include natural polysaccharides, and which may exhibit advantageous properties such as good conductivity of about 1 mS/cm to about 85 mS/cm, a high degree of stretchability of about 1000% to about 5500%, and self-healing capability, evidenced by the conductive hydrogel maintaining a storage modulus of about 15 kPa to about 60 kPa over a plurality of alternating low-high shear strain cycles. Methods of producing such conductive hydrogels, and devices containing the same, are also provided.

As used herein, the term “about” means plus or minus 10% of the numerical value of the number with which it is being used. For example, “about 50%” means in the range of 45-55% and also includes exactly 50%. Where any value is described herein as modified by the term “about,” the exact value is also disclosed.

As used herein, the term “self-healing” refers to materials that are capable of automatically repairing damage sustained by physical, chemical, or thermal forces.

As used herein, the term “self-sensing” refers to materials with the ability to detect micro- or nanoscale damage by observing a change in one of its properties, such as color, electrical properties, and other indicators.

As used herein, the term “graphene nanoplatelets” refers to graphene having a plurality of layers with a total thickness of about 3 nm to about 100 nm.

As used herein, the term “stretchability” refers to how much an object may be stretched without breaking. Stretchability is defined as the length of the conductive hydrogel after stretching divided by the length of the conductive hydrogel prior to stretching, multiplied by 100 to obtain a percentage.

This disclosure describes conductive hydrogels which may include a polysaccharide. The polysaccharide may include apple fibers, xanthan gum, Arabic gum, sodium carboxymethyl cellulose, konjac gum, guar gum, microfibrillated cellulose, gelatin, alginate, or combinations thereof. In embodiments, the conductive hydrogel includes one of the aforementioned polysaccharides. In embodiments, the conductive hydrogel includes two or more of the aforementioned polysaccharides. In embodiments where two or more polysaccharides are included in the conductive hydrogel, each individual polysaccharide may be included in equivalent or different amounts such that the total amount of polysaccharide adds up to about 5 wt. % to about 100 wt. %.

In embodiments, the conductive hydrogel includes about 5 wt. % to about 100 wt. % polysaccharide. For example, the conductive hydrogel may include about 5 wt. %, about 10 wt. % about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 100 wt. % polysaccharide, or any value contained within a range formed by any two of the preceding values.

In embodiments, the conductive hydrogel further includes about 1 wt. % to about 5 wt. % of a filler. For example, the conductive hydrogel may include about 1 wt. %, about 1.5 wt. %, about 2 wt. %, about 2.5 wt. %, about 3 wt. %, about 3.5 wt. %, about 4 wt. %, about 4.5 wt. %, about 5 wt. %, or any value contained within a range formed by any two of the preceding values.

In embodiments, the filler includes activated carbon black (ACB), reduced graphene oxide (rGO), graphene nanoplatelets (GNP), or combinations thereof. In embodiments, the conductive hydrogel does not include a filler.

In embodiments, the conductive hydrogel further includes about 2 wt. % to about 15 wt. % of a crosslinker relative to polysaccharide content. In embodiments, the crosslinker includes citric acid, borax (also called sodium tetraborate decahydrate), calcium chloride, or combinations thereof. In embodiments, the conductive hydrogel does not include a crosslinker.

In embodiments, the conductive hydrogel further includes about 5 wt. % to about 95 wt. % of a polymer, which may include polyvinyl alcohol (PVA), polyacrylamide, polyurethane, poly(acrylic acid), poly(methacrylic acid), poly(3,4-ethylenedioxythiophene)-poly(styrene sulfonate), or combinations thereof. For example, the conductive hydrogel may include about 5 wt. %, about 10 wt. % about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. % polymer, or any value contained within a range formed by any two of the preceding values. In embodiments, the polymer is hydrophilic. In embodiments, the conductive hydrogel does not contain any of polyvinyl alcohol, polyacrylamide, polyurethane, poly(acrylic acid), poly(methacrylic acid), or poly(3,4-ethylenedioxythiophene)-poly(styrene sulfonate), such that there can be considered to be 0 wt. % of polyvinyl alcohol, polyacrylamide, polyurethane, poly(acrylic acid), poly(methacrylic acid), or poly(3,4-ethylenedioxythiophene)-poly(styrene sulfonate) included in the conductive hydrogel.

The conductive hydrogel of the present disclosure may include a polysaccharide, a filler, a crosslinker, a polymer, or combinations thereof, in any of the above-disclosed amounts such that the total composition of the conductive hydrogel equals 100 wt. %.

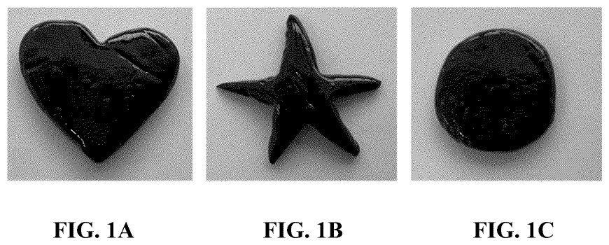

The shape of the conductive hydrogel of the present disclosure is not particularly limited. FIG. 1A, FIG. 1B, and FIG. 1C are a set of images of a conductive hydrogel according to an embodiment of the present disclosure, showing that the conductive hydrogel may be molded into a variety of shapes. The conductive hydrogel of the present disclosure may be molded into any shape, such as a circle, an oval, a square, a rectangle, a triangle, a star, or other shapes. The conductive hydrogel may be molded into any shape to suit a particular application, such as to cover a particular area of the body for use as a wound dressing or a health monitor. The conductive hydrogel may be molded to conform to skin or any other uneven or moving surface.

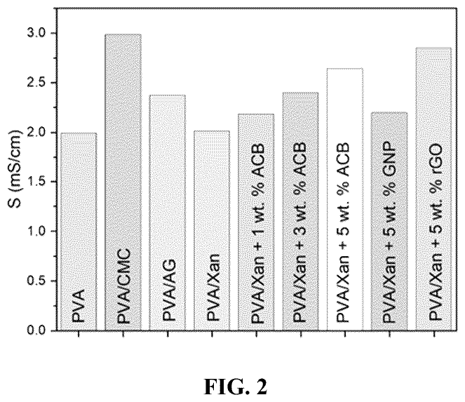

In embodiments, the conductive hydrogel exhibits a conductivity which may be measured. As will be familiar to those skilled in the art, conductivity may be measured in several ways, all of which are acceptable for determining the conductivity of the conductive hydrogel of the present disclosure. In embodiments, conductivity in represented in milliseimens per centimeter, and is abbreviated as mS/cm. In embodiments, the conductive hydrogel exhibits a conductivity of about 1 mS/cm to about 85 mS/cm, for example about 1 mS/cm, about 2 mS/cm, about 2.5 mS/cm, about 3 mS/cm, about 3.5 mS/cm, about 4 mS/cm, about 5 mS/cm, about 10 mS/cm, about 15 mS/cm, about 20 mS/cm, about 25 mS/cm, about 30 mS/cm, about 35 mS/cm, about 40 mS/cm, about 45 mS/cm, about 50 mS/cm, about 55 mS/cm, about 60 mS/cm, about 65 mS/cm, about 70 mS/cm, about 75 mS/cm, about 80 mS/cm, about 85 mS/cm, or any value contained within a range formed by any two of the preceding values. FIG. 2 is a bar graph showing the conductivity for various conductive hydrogels according to embodiments of the present disclosure. As shown in FIG. 2, conductive hydrogels which include a polysaccharide and/or a filler exhibit higher conductivities than hydrogels formed from a polymer such as PVA alone.

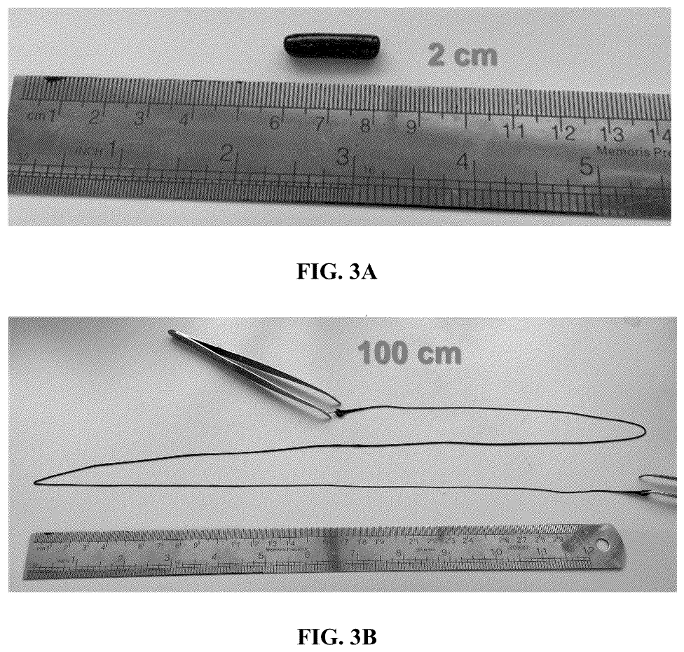

The conductive hydrogel of the present disclosure may be stretchable. The stretchability of the conductive hydrogel may be measured before and after stretching, wherein stretching is conducted by hand or by an instrument. Stretchability is defined as the length of the conductive hydrogel after stretching divided by the length of the conductive hydrogel prior to stretching, multiplied by 100 to obtain a percentage. In embodiments, the conductive hydrogel exhibits a stretchability of about 1000% to about 5500%, for example about 1000%, about 1500%, about 2000%, about 2500%, about 3000%, about 3500%, about 4000%, about 4500%, about 5000%, about 5500%, or any value contained within a range formed by any two of the preceding values. FIG. 3A and FIG. 3B are a set of images of a conductive hydrogel according to an embodiment of the present disclosure, demonstrating that the conductive hydrogel shown in FIG. 3A may be stretched to over 5000% its original length as shown in FIG. 3B.

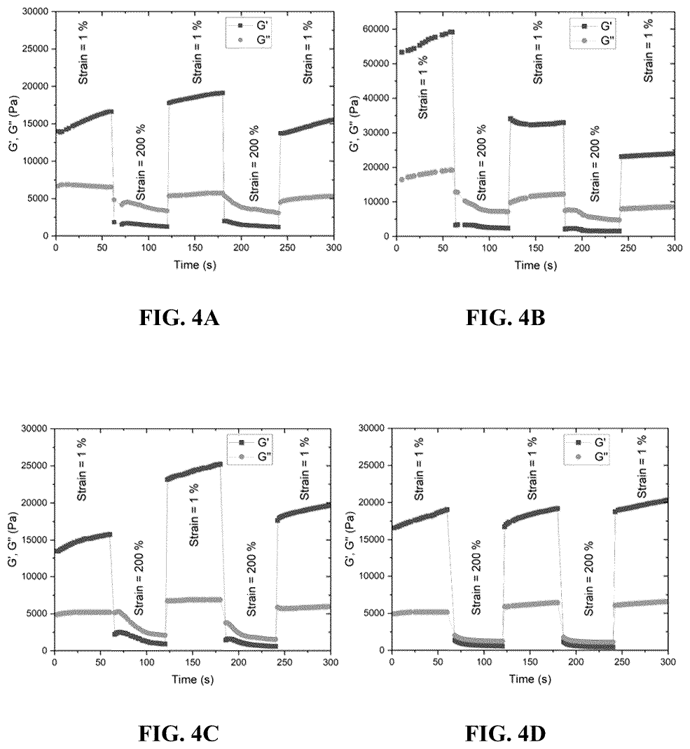

In embodiments, the conductive hydrogel exhibits an ability for self-healing. The self-healing ability of a material may be measured in several ways, including by evaluating the storage modulus (G′) and loss modulus (G″) over multiple cycles of low and high shear strain, alternating the amplitude between 1% and 200%. The 200% strain causes the material to undergo damage, and the 1% allows self-healing and recovery of mechanical properties. A reduction in G′ at high strain (200%) indicates the strain on the conductive hydrogels, and a return to high G′ at low strain (1%) indicates good self-healing ability and that the material recovers, as shown in FIG. 4A, FIG. 4B, FIG. 4C, and FIG. 4D. FIG. 4A, FIG. 4B, FIG. 4C, and FIG. 4D are plots demonstrating the self-healing ability of conductive hydrogels according to embodiments of the present disclosure, wherein shear strain is alternated between 1% and 200% over five cycles. A G′ value that decreases with each subsequent cycle, such as in FIG. 4B, indicates that the material has a lower ability for self-healing than a material which returns to the same high G′ value at low strain with each cycle, such as in FIG. 4D. FIG. 4A and FIG. 4C represent hydrogels that exhibit change in G′ with each cycle, though not as significant as that in FIG. 4B.

In embodiments, the conductive hydrogel exhibits a storage modulus of about 15 kPa to about 60 kPa over a plurality of alternating low-high shear strain cycles. For example, the conductive hydrogel may exhibit a storage modulus of about 15 kPa, about 20 kPa, about 25 kPa, about 30 kPa, about 35 kPa, about 40 kPa, about 45 kPa, about 50 kPa, about 55 kPa, about 60 kPa, or any value contained within a range formed by any two of the preceding values. In embodiments, the conductive hydrogel exhibits a storage modulus that remains substantially constant over multiple cycles of high and low strain. In embodiments, the conductive hydrogel of the present disclosure exhibits a storage modulus at low strain in subsequent cycles that is less than about 10 kPa lower than the storage modulus at low strain during the initial cycle. For example, the storage modulus at low strain in subsequent cycles may be less about 10 kPa, less than about 9 kPa, less than about 8 kPa, less than about 7 kPa, less than about 6 kPa, less than about 5 kPa, less than about 4 kPa, less than about 3 kPa, less than about 2 kPa, less than about 1 kPa lower, or any value contained within a range formed by any two of the preceding values, than the initial storage modulus at low strain.

In embodiments, there is provided a device which includes the conductive hydrogel according to any of the embodiments disclosed herein. The device may function as a strain sensor, a wound dressing, a health monitor, a self-healing material, or combinations thereof. In embodiments, the device may be connected to a computer.

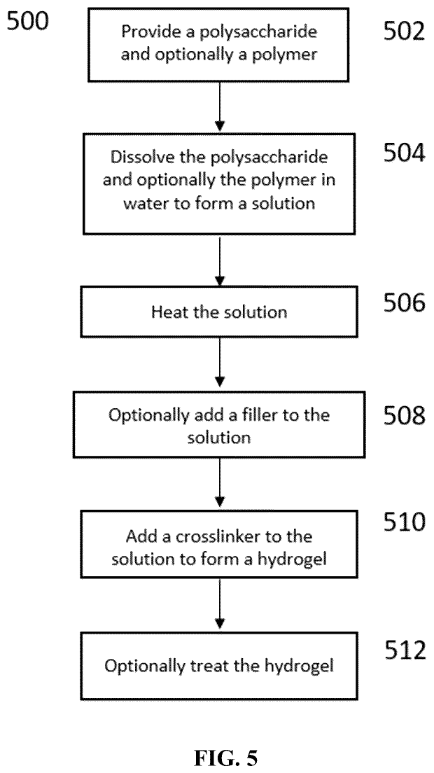

In one aspect of the present disclosure, there is provided a method of preparing conductive hydrogels. FIG. 5 is a flow chart of a method of making a conductive hydrogel, according to an embodiment of the present disclosure. The method 500 may include steps of providing a polysaccharide and optionally a polymer 502, dissolving the polysaccharide and optionally the polymer in water to form a solution 504, heating the solution 506, optionally adding a filler to the solution 508, adding a crosslinker to the solution to form a hydrogel 510, and optionally treating the hydrogel 512.

In embodiments, step 502 of providing a polysaccharide and optionally a polymer includes providing a polysaccharide only, such that the polymer is omitted. In embodiments, step 502 includes providing a polysaccharide and further includes providing a polymer. In embodiments, the polysaccharide may include apple fibers, xanthan gum, Arabic gum, sodium carboxymethyl cellulose, konjac gum, guar gum, microfibrillated cellulose, gelatin, alginate, or combinations thereof. In embodiments, the polymer includes polyvinyl alcohol (PVA), polyacrylamide, polyurethane, poly(acrylic acid), poly(methacrylic acid), poly(3,4-ethylenedioxythiophene)-poly(styrene sulfonate), or combinations thereof.

In embodiments, the method 500 includes step 504 of dissolving the polysaccharide and optionally the polymer in water to form a solution. The amount of polysaccharide contained within the solution is not particularly limited. For example, the solution may include about 1 wt. % to about 10 wt. % of polysaccharides relative to the total weight of the solution, such as about 1 wt. %, about 2 wt. %, about 3 wt. %, about 4 wt. %, about 6 wt. %, about 7 wt. %, about 8 wt. %, about 9 wt. %, about 10 wt. %, or any value contained within a range formed by any two of the preceding values. The concentration of the solution may be adjusted according to the solubility of the polysaccharide. In embodiments, the method 500 includes step 504 dissolving the polysaccharide in water to form a solution and further includes dissolving the polymer in the solution. In embodiments, the polysaccharide and the polymer are dissolved separately and then combined into one solution. In other embodiments, the polysaccharide and the polymer are dissolved together. In embodiments, the polymer is omitted.

In embodiments, the method 500 includes a step of heating the solution 506. Heating the solution 506 may include heating the solution to a temperature of about 50° C. to about 100° C., for example, about 50° C., about 55° C., about 60° C., about 65° C., about 70° C., about 75° C., about 80° C., about 85° C., about 90° C., about 95° C., about 100° C., or any value contained within a range formed by any two of the preceding values. The step of heating the solution 506 may also include stirring the solution, such as with a mechanical stirrer.

In embodiments, the method 500 includes a step of adding a filler to the solution 508. In embodiments, the filler includes activated carbon black (ACB), reduced graphene oxide (rGO), graphene nanoplatelets (GNP), or combinations thereof. In embodiments, the filler is added to the solution in such an amount that the amount of filler in the conductive hydrogel is about 1 wt. % to about 5 wt. %, such as about 1 wt. %, about 1.5 wt. %, about 2 wt. %, about 2.5 wt. %, about 3 wt. %, about 3.5 wt. %, about 4 wt. %, about 4.5 wt. %, about 5 wt. %, or any value contained within a range formed by any two of the preceding values. In embodiments, step 508 is not performed, such that the filler is omitted.

In embodiments, the method 500 includes a step of adding a crosslinker to the solution to form a hydrogel 510. In embodiments, the crosslinker includes citric acid, borax (also called sodium tetraborate decahydrate), calcium chloride, or combinations thereof. The amount of the crosslinker in the solution may be about 0.05 M to about 0.15 M, for example about 0.05 M, about 0.06 M, about 0.07 M, about 0.08 M, about 0.09 M, about 0.10 M, about 0.11 M, about 0.12 M, about 0.13 M, about 0.14 M, about 0.15 M, or any value contained within a range formed by any two of the preceding values.

In embodiments, the method 500 includes a step of treating the hydrogel 512. In embodiments, treating the hydrogel includes freeze drying the hydrogel, contacting the hydrogel with a solution, or combinations thereof. In embodiments that include both freeze drying the hydrogel and contacting the hydrogel with a solution, the order in which these treatments are performed is not limited. For example, in embodiments, the hydrogel may be first freeze dried and then contacted with a solution, and in other embodiments the hydrogel may be first contacted with a solution and then freeze dried. In embodiments, the solution includes lithium chloride, potassium chloride, sulfuric acid, potassium hydroxide, sodium sulfate, or combinations thereof. In embodiments, step 512 is omitted, such that the hydrogel is not treated as described herein.

The embodiments and aspects disclosed herein may be combined in any fashion to create new embodiments.

### Examples

Conductive hydrogels were prepared using methods as described herein. More precisely, two separate solutions, 25 wt. % solution of PVA (2 g in 8 g of H₂O) and 3 wt. % of polysaccharides, Na-CMC or Xan, (0.167 g in 5.33 g of H₂O) were prepared at 80° C. at 600 rpm. In the case of Arabic gum, 0.167 g of Arabic gum was directly added to the PVA solution since the Arabic gum solution showed a high loss in viscosity when heated. After complete dissolution of PVA and polysaccharides, the two solutions were mixed on a mechanical stirrer at 80° C. at 600 rpm. Then, 1-5 wt. % of graphene-based fillers ACB/GNP/rGO (relative to PVA solid content) were added to the mixture and stirred for the next 10 min. Finally, a borax solution (0.12 M, 3.33 ml) was added dropwise into the prepared mixture with vigorous mixing until the conductive nanocomposite hydrogel was prepared.

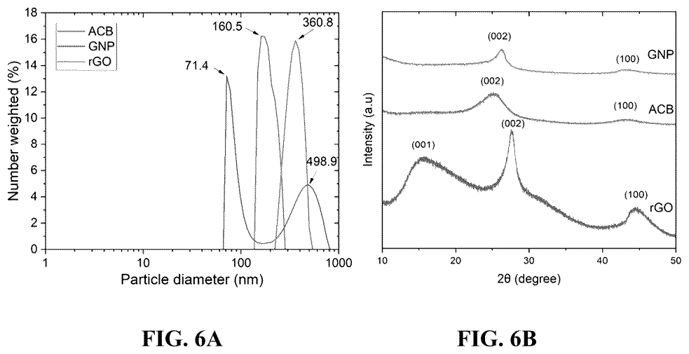

The size distribution of ACB, GNP, and rGO was determined by the dynamic light scattering (DLS) method using a particle analyzer LiteSizer 500 from Anton Paar, Germany. The measurements were performed in a back-scattering configuration at 25° C. in isopropanol. FIG. 6A shows particle size distribution of samples according to embodiments of the present disclosure. The lowest diameter was found for GNP particles (71.4 nm), where the second peak can be attributed to the appearance of agglomerates in isopropanol, which is expected due to the high affinity of nanoparticles to form aggregates due to the weak dispersibility of graphene on isopropanol.

Powder X-ray diffraction (XRD) tests were recorded on a Bruker D8 Advance instrument (Bruker, Germany). Cu-Kα radiations were employed as an X-ray source (λ=0.154 nm). The test was run in 0-60° range at room temperature, with an 0.01° increment and 0.5 s/step. FIG. 6B shows XRD patterns of three samples according to embodiments of the present disclosure, ACB, GNP, and rGO.

The surface atomic composition of graphite-based fillers was examined by XPS using a PHI VersaProbe 5000 Scanning x-ray Photoelectron Spectrometer with an Mg Ka x-ray source (1100 eV). A monochromated Mg X-ray source (1100 eV) was used as a probe for the experiments. The X-ray beam power was 50.17 W with the step size of 0.05 eV and detector pass energy of 280 eV. E-neutralizer (1V) and I-neutralizer (0.11 kV Ar⁺ ion) were implemented during the experiment. The compositions were calculated by using the area under the high-resolution and weighted with the respective sensitivity factors for each elemental species. Peaks were calibrated using the C1 s as a reference at 284.6 eV. The software Casa Xps was used for curve fitting and calibration.

The dynamic mechanical properties of conductive nanocomposite hydrogels were analyzed on a rheometer at 25° C. (Brookfield RSO oscillatory Rheometer supplied by Venktron, UAE) in conical plate mode. Amplitude sweep measurements were performed in a shear strain mode with a start amplitude of 0.06%, end amplitude of 200.00%, frequency of 10 Hz, and 30 steps, and they showed the yield and flow point of hydrogels. Time-dependent behavior was evaluated in 5 blocks in a shear strain mode, alternating the amplitude of 1% and 200% at 10 Hz during 60 s each block. Frequency Sweep measurements were also performed in a shear strain mode with 0.10% amplitude, starting from 0.10 Hz until 10 Hz and 30 steps.

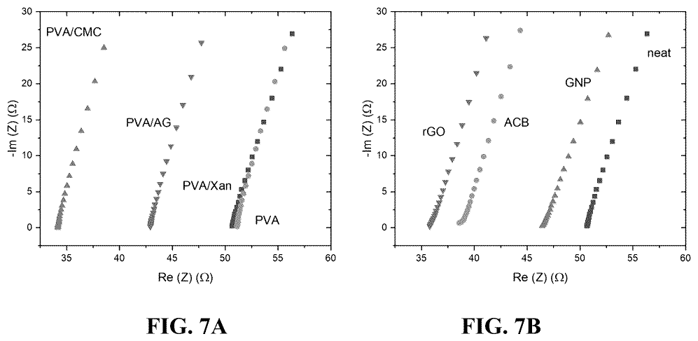

The electrochemical impedance spectra (EIS) were recorded in the frequency range of 100 kHz-10 mHz with a potential amplitude of 10 mV on BioLogic's VSP-300 channel potentiostat. To gain an insight into the conductivity of synthesized hydrogels, the electrochemical impedance spectroscopy (EIS) with different polysaccharide modifiers, fillers, and amounts of fillers was performed as shown in FIG. 7A and FIG. 7B. FIG. 7A and FIG. 7B are Nyquist diagrams of conductive hydrogels according to embodiments of the present disclosure. The results in FIG. 7A, investigating the effect of different polysaccharide modifiers, show that PVA and PVA/Xan have similar conductivity values, while PVA/AG and PVA/CMC showed 16.3% and 33.4% higher values than PVA, respectively. The reason for that, without wishing to be bound by theory, lies in the fact that both polysaccharide modifiers AG and CMC possess ionic conductivity as their structure includes salts. The values for 1 wt. % and 3 wt. % of ACB added are much higher than neat PVA/Xan, for 17.7% and 16.7%, respectively. The absence of improvement by the addition of 3 wt. % ACB may be be attributed to aggregates since no homogenization technique was used. Nevertheless, the conductivity further increased with further addition of ACB since it was 32.3% higher than PVA/Xan in the case of the PVA/Xan+5 wt. %. FIG. 7B shows Nyquist diagrams for neat PVA/Xan and conductive nanocomposite hydrogels with 5 wt. % of different fillers (GNP, ACB, and rGO). The highest conductivity (S) was found for rGO fillers. S for rGO fillers was 29.8% and 6.5% higher than GNP and ACB, respectively. Very similar electrochemical properties of ACB and rGO suggest a high potential for using ACB as a cost-effective conductive filler.

TABLE 1

| | | Wt. % of | Wt. % | |
|---|---|---|---|---|
| | Polysac- | polysac- | of | |
| Sample | charide | charide | PVA | Filler |
| 1 | None | 0 | 100 | None |
| 2 | Xan | 7.7 | 92.3 | None |
| 3 | Xan | 7.7 | 92.3 | 1 wt. % ACB |
| 4 | Xan | 7.7 | 92.3 | 3 wt. % ACB |
| 5 | Xan | 7.7 | 92.3 | 5 wt. % ACB |
| 6 | Xan | 7.7 | 92.3 | 5 wt. % GNP |
| 7 | Xan | 7.7 | 92.3 | 5 wt. % rGO |
| 8 | Xan | 7.7 | 92.3 | 10 wt. % ACB |
| 9 | AF | 7.7 | 92.3 | None |
| 10 | AF | 7.7 | 92.3 | 5 wt. % ACB |
| 10a | AF | 7.7 | 92.3 | 5 wt. % GNP |
| 11 | AG | 7.7 | 92.3 | None |
| 12 | AG | 7.7 | 92.3 | 5 wt. % ACB |
| 13 | Na-CMC | 7.7 | 92.3 | None |
| 14 | Na-CMC | 100 | 0 | None |
| 15 | Na-CMC | 100 | 0 | 5 wt. % ACB |
| 16 | Na-CMC | 7.7 | 92.3 | 5 wt. % ACB |
| 17 | KG | 7.7 | 92.3 | None |
| 18 | KG | 7.7 | 92.3 | 5 wt. % ACB |
| 19 | GG | 7.7 | 92.3 | None |
| 20 | GG | 7.7 | 92.3 | 5 wt. % ACB |
| 21 | MFC | 7.7 | 92.3 | None |
| 22 | Xan, AF | 100 | 0 | None |
| 23 | Xan, AF | 100 | 0 | 5 wt. % ACB |

After freeze drying and absorption of different electrolytes

| | | | | |
|---|---|---|---|---|
| 24 | AF (H₂SO₄) | 7.7 | 92.3 | None |
| 25 | AF (LiCl) | 7.7 | 92.3 | None |
| 26 | GG (H₂SO₄) | 7.7 | 92.3 | None |
| 27 | GG (LiCl) | 7.7 | 92.3 | None |

TABLE 1 shows a range of conductive hydrogels prepared according to methods of the present disclosure, including various amounts and combinations of polysaccharide and fillers. Samples 24-27 further include freeze drying the hydrogel and contacting the hydrogel with a solution as described in parentheses.

TABLE 2

| | | | G′ at 1% | G′ at 1% | G′ at 1% |
|---|---|---|---|---|---|
| | Con- | Stretch- | strain | strain | strain |
| | ductivity | ability | Cycle 1 | Cycle 2 | Cycle 3 |
| Sample | (mS/cm) | (%) | (kPa) | (kPa) | (kPa) |
| 1 | 1.97 | 1000 | 16.6 | 19.1 | 15.5 |
| 2 | 1.98 | 5000 | 19.0 | 19.2 | 20.2 |
| 3 | 2.33 | 5100 | — | — | — |
| 4 | 2.31 | 5000 | — | — | — |
| 5 | 2.62 | 5500 | — | — | — |
| 6 | 2.15 | 4800 | — | — | — |
| 7 | 2.79 | 4300 | — | — | — |
| 8 | — | 5100 | — | — | — |
| 9 | 2.86 | 4600 | 49.9 | 13.5 | 9.7 |
| 10 | — | — | 41.0 | 15.3 | 9.0 |
| 10a | — | — | 17.8 | 8.8 | 6.2 |
| 11 | 2.36 | — | 59.1 | 32.9 | 23.7 |
| 13 | 2.96 | — | 15.8 | 25.2 | 19.6 |
| 16 | — | — | 26.1 | 26.1 | 25.1 |
| 17 | 2.18 | — | 29.6 | 10.7 | 6.0 |
| 18 | — | — | 31.0 | 7.5 | 5.5 |
| 19 | 3.70 | — | 28.0 | 14.0 | 9.5 |
| 20 | — | — | 33.0 | 12.7 | 9.9 |

After freeze drying and absorption of different electrolytes

| | | | | | |
|---|---|---|---|---|---|
| 24 | 81.86 | — | — | — | — |
| 25 | 2.74 | — | — | — | — |
| 26 | 57.25 | — | — | — | — |

TABLE 2 shows the properties associated with a selection of the samples introduced in TABLE 1. The conductivity, stretchability, and storage modulus (G′) were measured according to methods described herein.

As shown in TABLE 2, the conductivity of samples 1-19 ranged from about 1 mS/cm to about 4 mS/cm. Samples 24 and 26, however, demonstrate a conductivity value from about 57 mS/cm to about 82 mS/cm. Without wishing to be bound by theory, it is believed that freeze drying the hydrogels and contacting them with an electrolyte solution, particularly sulfuric acid as in samples 24 and 26, may increase the conductivity of the hydrogels of the present disclosure.

While a number of the samples in TABLE 2 exhibit strong G′ values initially, samples 2, 13, and 16 exhibit a very consistent storage modulus across multiple cycles. Sample 16, without wishing to be bound by theory, demonstrates the benefit of including both a polysaccharide and a filler on the self-healing ability of the hydrogels.

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 compounds refers to groups having 1, 2, 3, 4, or 5 compounds, and so forth.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A conductive hydrogel, comprising:
5 wt. % to 100 wt. % of a polysaccharide,
wherein the conductive hydrogel exhibits a conductivity of 1 mS/cm to 85 mS/cm and a stretchability of 1000% to 5500%,
wherein the conductive hydrogel exhibits self-healing.

2. The conductive hydrogel of claim 1, wherein the polysaccharide comprises apple fibers, xanthan gum, Arabic gum, sodium carboxymethyl cellulose, konjac gum, guar gum, microfibrillated cellulose, gelatin, alginate, or combinations thereof.

3. The conductive hydrogel of claim 1, wherein the conductive hydrogel comprises 10 wt. % to 30 wt. % polysaccharide.

4. The conductive hydrogel of claim 1, wherein the conductive hydrogel comprises 50 wt. % to 100 wt. % polysaccharide.

5. The conductive hydrogel of claim 1, further comprising 1 wt. % to 5 wt. % of a filler.

6. The conductive hydrogel of claim 5, wherein the filler comprises activated carbon black, reduced graphene oxide, graphene nanoplatelets, or combinations thereof.

7. The conductive hydrogel of claim 1, further comprising 2 wt. % to 15 wt. % of a crosslinker.

8. The conductive hydrogel of claim 7, wherein the crosslinker comprises citric acid, borax, calcium chloride, or combinations thereof.

9. The conductive hydrogel of claim 1, further comprising 5 wt. % to 95 wt. % polyvinyl alcohol, polyacrylamide, polyurethane, poly(acrylic acid), poly(methacrylic acid), poly(3,4-ethylenedioxythiophene)-poly(styrene sulfonate), or combinations thereof.

10. The conductive hydrogel of claim 1, wherein the conductive hydrogel exhibits a conductivity of 2.5 mS/cm to 80 mS/cm.

11. The conductive hydrogel of claim 1, wherein the conductive hydrogel exhibits a stretchability of 3000% to 5500%.

12. The conductive hydrogel of claim 1, wherein the conductive hydrogel exhibits a storage modulus of 15 kPa to 60 kPa over a plurality of alternating low-high shear strain cycles.

13. A device, comprising the conductive hydrogel of claim 1, wherein the device functions as a strain sensor, a wound dressing, a health monitor, a self-healing material, or combinations thereof.

14. The device of claim 13, wherein the device is connected to a computer.

15. A method of making a conductive hydrogel, comprising:
providing a polysaccharide,
dissolving the polysaccharide in water to form a solution,
heating the solution, and
adding a crosslinker to the solution to form a hydrogel.

16. The method of claim 15, wherein the polysaccharide comprises apple fibers, xanthan gum, Arabic gum, sodium carboxymethyl cellulose, konjac gum, guar gum, microfibrillated cellulose, gelatin, alginate, or combinations thereof.

17. The method of claim 15, further comprising dissolving a polymer in the solution, wherein the polymer comprises polyvinyl alcohol, polyacrylamide, polyurethane, poly(acrylic acid), poly(methacrylic acid), poly(3,4-ethylenedioxythiophene)-poly(styrene sulfonate), or combinations thereof.

18. The method of claim 15, wherein heating the solution comprises heating the solution to a temperature of 50° C. to 100° C.

19. The method of claim 15, further comprising adding a filler to the solution, wherein the filler comprises activated carbon black, reduced graphene oxide, graphene nanoplatelets, or combinations thereof.

20. The method of claim 15, wherein the crosslinker comprises citric acid, borax, calcium chloride, or combinations thereof.

21. The method of claim 15, further comprising treating the hydrogel, wherein treating the hydrogel comprises freeze drying the hydrogel, contacting the hydrogel with a solution, or combinations thereof.

22. The method of claim 21, wherein the solution comprises lithium chloride, potassium chloride, sulfuric acid, potassium hydroxide, sodium sulfate, or combinations thereof.
